(19) United States
(12) Patent Application Publication
(10) Pub. No.: US 2025/0166833 A1
(43) Pub. Date: May 22, 2025

(54) APPARATUS, METHOD, AND PROGRAM THAT PROVIDE TOTAL HEALTHCARE SOLUTION SERVICE THROUGH COMPANION ANIMAL HAIR ANALYSIS

(71) Applicant: PRECISION AND PERSONALIZED MEDICINE INCORPORATION (PPMI), Goyang-si, Gyeonggi-do (KR)

(72) Inventors: Tai Gyu LEE, Seoul (KR); Yujin EOM, Seoul (KR)

(73) Assignee: PRECISION AND PERSONALIZED MEDICINE INCORPORATION (PPMI), Goyang-si, Gyeonggi-do (KR)

(21) Appl. No.: 18/840,721

(22) PCT Filed: Feb. 3, 2023
(86) PCT No.: PCT/KR2023/001623

§ 371 Date: Oct. 10, 2024

(30) Foreign Application Priority Data
Feb. 22, 2022 (KR) 10-2022-0022938

(51) Int. Cl. G16H 50/30; G01N 33/483; G16H 10/60; G16H 20/60; H01J 49/10

(52) U.S. Cl. CPC G16H 50/30; G01N 33/4833; G16H 10/60; G16H 20/60; H01J 49/105

(57) ABSTRACT

An apparatus for providing a companion animal menu platform service including a companion animal information receiving unit for receiving companion animal information on a companion animal of a user from a user terminal of the user; a nutrition information generating unit for determining, for the companion animal, daily intake calories, an intake-restricted material, an intake-restricted nutrient, a first protein ratio, a first fat ratio, and a first fiber ratio, contained in food, on the basis of the companion animal information; a material determining unit for determining a plurality of materials on the basis of the companion animal information, determining, as a plurality of first preliminary materials, the remaining materials excluding a material corresponding to the intake-restricted material, among the materials, determining, as a plurality of second preliminary materials, the remaining materials excluding first preliminary material containing the intake-restricted nutrient, among the first preliminary materials.

### TECHNICAL FIELD

The present invention relates to an apparatus, method, and program for providing a total healthcare solution service through companion animal hair analysis.

### BACKGROUND ART

Unless otherwise indicated herein, contents described in this section are not intended to be prior art to claims of this application, and its inclusion in this section is not intended to be deemed prior art.

The number of people raising companion animals is gradually increasing, and awareness of companion animals as family rather than simply objects to raise is increasing.

Accordingly, interest in the health of a companion animal is increasing, and in particular, interest in services that can examine the health status of a companion animal is increasing.

However, most users of a companion animal's health checkup service feel burdened by the cost, and thus, there is a need for technology that can accurately check the health of a companion animal while incurring a relatively low cost.

### DISCLOSURE

### Technical Problem

Therefore, the present invention has been made in view of the above problems, and it is one object of the present invention to provide an apparatus, method, and program for providing a total healthcare solution service that examines the health status of a user's companion animal related to nutritional minerals and the health status of the companion animal related to hazardous heavy metals based on analysis results of the hair of the user's companion animal and provides the examination results to the user.

It is another object of the present invention to provide an apparatus, method, and program for providing a total healthcare solution service that determines nutritional minerals that need to be consumed preferentially and nutritional minerals that need to be avoided for the user's companion animal, and provides information about the determined nutritional minerals to the user.

It is yet another object of the present invention to provide an apparatus, method, and program for providing a total healthcare solution service that determines food suitable for the user's companion animal and provides information about the determined food to the user.

### Technical Solution

In accordance with an aspect of the present invention, the above and other objects can be accomplished by the provision of an apparatus for providing a total healthcare solution service through companion animal hair analysis.

The apparatus may include a hair analysis result reception part configured to receive a hair analysis result that includes a mineral level of each of a plurality of nutritional minerals included in hair of a user's companion animal and a heavy metal level of each of a plurality of harmful heavy metals included in the hair; and a solution analysis part configured to compare the mineral level of each of the nutritional minerals with a standard range corresponding to each of the nutritional minerals so as to classify the nutritional mineral into a management-requiring nutritional mineral or a standard nutritional mineral, and to compare the heavy metal level of each of the harmful heavy metals with the safe level corresponding to each of the harmful heavy metals so as to classify the harmful heavy metals into a harmful heavy metal to be cautious of or a safe-level harmful heavy metal.

In addition, the solution analysis part may determine a nutritional mineral, whose mineral level is not included in the standard range, among the nutritional minerals as the management-requiring nutritional mineral, determine a nutritional mineral, whose mineral level is included in the standard range, among the nutritional minerals as the standard nutritional mineral, determine a harmful heavy metal, whose heavy metal level is equal to or higher than the safe level, among the harmful heavy metals as the harmful heavy metal to be cautious of, and determine a harmful heavy metal, whose heavy metal level is smaller than the safe level, among the harmful heavy metals as the safe-level harmful heavy metal.

In addition, the apparatus may further include a companion animal information reception part configured to receive companion animal information including information on age, neutering, weight, personality, species, breed, body type, activity information, disease, staple diet, and snacks of the companion animal from a terminal of the user, receive a first survey result including a response to a first survey that includes a plurality of questions about each of the harmful heavy metals, and receive a second survey result including a response to a second survey including a plurality of questions about each of the nutritional minerals; and a recommendation test decision part configured to input the companion animal information and the first survey result as input values into a first machine-learning model that has been previously learned, obtain a first possibility that an abnormality related to the harmful heavy metals exits from the first machine-learning model, input the companion animal information and the second survey result as input values into a second machine-learning model that has been previously learned, obtain a second possibility that an abnormality related to the nutritional minerals exists from the second machine-learning model, and determine a test to be recommended to the user among a first test, second test and third test that have been preset based on the first possibility and the second possibility.

In addition, the first test may test a health status of the companion animal related to the nutritional minerals, the second test may test a health status of the companion animal related to the harmful heavy metals, and the third test may test a health status of the companion animal related to the nutritional minerals and the harmful heavy metals.

In addition, the solution analysis part may input information on the age, neutering status, weight, personality, species, breed and body shape included in the companion animal information as input values into a third machine-learning model that has been previously learned and obtain the safe level of each of the harmful heavy metals from the third machine-learning model, and input information on the age, neutering status, weight, personality, species, breed and body shape included in the companion animal information as input values into a fourth machine-learning model that has been previously learned and obtain the standard range of each of the nutritional minerals from the fourth machine-learning model.

In addition, the nutritional minerals may include calcium, sodium, potassium, phosphorus, magnesium, copper, zinc, iron, manganese, chromium and selenium, and the harmful heavy metals include mercury, arsenic, cadmium, lead, aluminum, nickel and uranium.

In addition, the hair analysis result may include a plurality of first indices including a ratio of the mineral level of the magnesium to the mineral level of the calcium, a ratio of the mineral level of the phosphorus to the mineral level of the calcium, a ratio of the mineral level of the potassium to the mineral level of the sodium, a ratio of the mineral level of the copper to the mineral level of the zinc, a ratio of the mineral level of the magnesium to the mineral level of the sodium and a ratio of the mineral level of the potassium to the mineral level of the calcium.

In addition, the hair analysis result may include a plurality of second indices including a ratio of the heavy metal level of the mercury to the mineral level of the zinc, a ratio of the heavy metal level of the mercury to the mineral level of the selenium, a ratio of the heavy metal level of the lead to the mineral level of the calcium, a ratio of the heavy metal level of the lead to the mineral level of the iron, a ratio of the heavy metal level of the lead to the mineral level of the zinc and a ratio of the heavy metal level of the cadmium to the mineral level of the zinc.

In addition, the solution analysis part may determine a first index, which is not included in a preset reference range, among the first indices as a first caution index, determine a first index, which is included in the reference range, among the first indices as a first safety index, determine a second index, which is lower than a present safe level, among the second indices as a second caution index, and determine a second index, which is greater than the safe level, among the second indices as the second safety index.

In addition, the solution analysis part may calculate an intake necessity level corresponding to each of the nutritional minerals based on the mineral level, the standard range, the first indices and the reference range, list the nutritional minerals in order of high need for intake, and determine the first to Nth nutritional minerals among the nutritional minerals as intake-required nutritional minerals.

In addition, the solution analysis part may determine a value, obtained by multiplying a standard value and weighted value that have been set for each of the nutritional minerals, as the intake necessity level for each of the nutritional minerals, determines a value, obtained by adding a correction value to a value obtained by dividing a first difference value between the minimum value of the standard range and the mineral level by a second difference value between the maximum value of the standard range and the minimum value thereof, as the standard value of the nutritional mineral when the mineral level is smaller than the minimum value of the standard range, determine a value, obtained by dividing a third difference value between the median value of the standard range and the mineral level thereof by the second difference value between the maximum value of the standard range and the minimum value thereof, as the standard value of the nutritional mineral when the mineral level is smaller than the median value of the standard range and greater than the minimum value thereof, determine a minimum value of a value that can be derived by dividing the third difference value by the second difference value as the standard value of the nutritional minerals when the mineral level is greater than the median value of the standard range, determine the weighted value of the nutritional mineral corresponding to a numerator of the first index to be a value greater than 1 and determine the weighted value of the nutritional minerals corresponding to a denominator of the first index to be 1 when the first index is smaller than the minimum value of the reference range, determine the weighted value of the nutritional mineral corresponding to a denominator of the first index to be a value greater than 1 and the weighted value of the nutritional mineral corresponding to a numerator of the first index to be 1 when the first index is greater than the maximum value of the reference range, and determine the weighted value of each of the nutritional minerals corresponding to a denominator and numerator of the first index to be 1 when the first index is included in the reference range.

### Advantageous Effects

In accordance with an embodiment of the present invention, the health status of a companion animal related to nutritional minerals and harmful heavy metals is provided based on analysis results of the hair of the companion animal, so that the cost required for a health status checkup of the companion animal can be reduced.

In accordance with another embodiment of the present invention, information on nutritional minerals that a companion animal should preferentially consume, nutritional minerals that should be avoided from consumption, and foods containing nutritional minerals is provided to a user, so that the user can control the diet of the companion animal based on the provided information.

In accordance with yet another embodiment of the present invention, information on food suitable for the health status of a companion animal is provided, so that a user can adjust the diet of the companion animal based on the provided information.

### DESCRIPTION OF DRAWINGS

FIG. 1 illustrates a schematic diagram of a system for providing a total healthcare solution service according to an embodiment.

FIG. 2 is a block diagram exemplarily illustrating a functional module of a service-providing apparatus according to FIG. 1.

FIG. 3 is a diagram conceptually illustrating a process of determining the type of test, recommended to a user's companion animal, by a recommendation test decision part according to FIG. 2.

FIG. 4 is a diagram conceptually illustrating a process of receiving a hair analysis result by a hair analysis result reception part according to FIG. 2.

FIG. 5 is a flowchart illustrating a process of determining harmful heavy metals requiring caution among harmful heavy metals and nutritional minerals requiring management among nutritional minerals by a solution analysis part according to FIG. 2.

FIG. 6 is a diagram conceptually illustrating a process where a solution analysis part according to FIG. 2 provides a user with a test result of harmful heavy metals and a test result of nutritional minerals.

FIG. 7 is a diagram conceptually illustrating a process where the solution analysis part according to FIG. 2 provides a test result of harmful heavy metals to a user.

FIG. 8 is a diagram conceptually illustrating a process where the solution analysis part according to FIG. 2 provides a user with a test result of nutritional minerals.

FIG. 9 is a flowchart illustrating a process of determining first and second indices, which require attention, among the first indices and second indices by the solution analysis part according to FIG. 2.

FIG. 10 is a diagram conceptually illustrating a process where test results of the first indices and second indices are provided to a user by the solution analysis part according to FIG. 2.

FIG. 11 is a flowchart illustrating a process of determining intake-required nutritional minerals by the solution analysis part according to FIG. 2.

FIG. 12 is a diagram conceptually illustrating a process of providing intake-required nutritional minerals and foods containing the intake-required nutritional minerals to a user by the solution analysis part according to FIG. 2.

FIG. 13 is a flowchart illustrating a process of determining intake caution-requiring nutritional minerals by the solution analysis part according to FIG. 2.

FIG. 14 is a flowchart illustrating a process of determining risk factors, which a user should be careful about, by a risk factor analysis part according to FIG. 2.

FIG. 15 is a flowchart illustrating a process of determining recommendation food by a recommendation food decision part according to FIG. 2.

FIG. 16 exemplarily illustrates a hardware configuration of the service-providing apparatus according to FIG. 1.

### BEST MODE

Since the present invention may be applied with various modifications and may have various embodiments, exemplary embodiments and drawings of the present invention are intended to be explained and exemplified. However, these exemplary embodiments and drawings are not intended to limit the embodiments of the present invention to particular modes of practice, and all changes, equivalents, and substitutes that do not depart from the spirit and technical scope of the present invention should be understood as being encompassed in the present invention. Like reference numerals refer to like elements in describing each drawing.

The terms such as “first,” “second,” “A” and “B” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present invention. The term “and/or” includes any or all combinations of one or more of the associated listed items.

It should be understood that when an element is referred to as being “connected to” or “coupled to” another element, the element may be directly connected or coupled to the other element or intervening elements may be present. In contrast, when an element is referred to as being “directly connected to” or “directly coupled to” another element, there are no intervening elements present.

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the present invention will be described in detail by explaining exemplary embodiments of the invention with reference to the attached drawings.

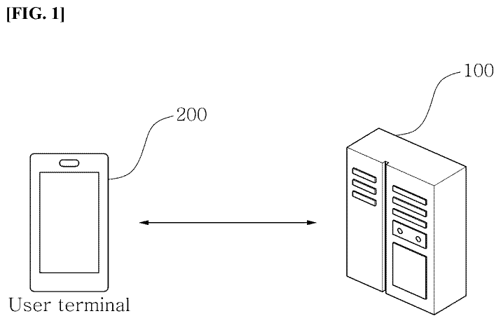

FIG. 1 illustrates a schematic diagram of a system for providing a total healthcare solution service according to an embodiment.

Referring to FIG. 1, the system for providing a total healthcare solution service includes a service-providing apparatus 100 and a user terminal 200.

The user terminal 200 is a terminal of a user who wants to use the total healthcare solution service, user's companion animal information and a survey result, which is a response to a preset survey, may be registered in the service-providing apparatus 100, and various functions of the total healthcare solution service may be used through the service-providing apparatus 100. In an embodiment, the companion animal information may include information about the age, neutering status, weight, personality, species, breed, body type, activity level, disease, stock, and snacks of a companion animal. In an embodiment, the survey may include a plurality of questions related to a plurality of preset harmful heavy metals. In an embodiment, the survey may include a plurality of questions related to a plurality of preset nutritional minerals.

Examples of the user terminal 200 include a communicable desktop computer, a laptop computer, a notebook, a smartphone, a tablet PC, a mobile phone, a smartwatch, a smart glass, an e-book reader, a portable multimedia player (PMP), a portable game console, a navigation device, a digital camera, a digital multimedia broadcasting (DMB) player, a digital audio recorder, a digital audio player, a digital video recorder, a digital video player, a personal digital assistant (PDA), and the like.

Specifically, the service-providing apparatus 100 may receive the companion animal information and survey result about the user's companion animal from the user terminal 200. The service-providing apparatus 100 may provide a user interface for selecting or entering the companion animal information about the user's companion animal to the user terminal 200. In addition, the service-providing apparatus 100 may provide a user interface for inputting a response to a survey to the user terminal 200.

In addition, the service-providing apparatus 100 may receive a hair analysis result, which is a result of analyzing the hair of the user's companion animal, through an input interface device included in the service-providing apparatus 100. In addition, the service-providing apparatus 100 may receive a hair analysis result, which is a result of analyzing the hair of the user's companion animal, from a separate server.

In addition, the service-providing apparatus 100 may compare the heavy metal level of each of a plurality of harmful heavy metals included in the hair analysis result with the safety range of each of the harmful heavy metals, and classify harmful heavy metals based on whether the heavy metal level is included in the safety range.

In addition, the service-providing apparatus 100 may compare the mineral value of each of a plurality of nutritional minerals included in the hair analysis result with the standard range of each of the nutritional minerals, and classify nutritional minerals based on whether the mineral value is included in the standard range.

In addition, the service-providing apparatus 100 may generate a harmful heavy metal test result based on the classification result of the harmful heavy metals, and may provide the generated harmful heavy metal test result to the user terminal 200.

In addition, the service-providing apparatus 100 may generate a nutritional mineral test result based on the classification result of the nutritional minerals, and may provide the generated nutritional mineral test result to the user terminal 200.

In addition, the service-providing apparatus 100 may compare each of the plurality first indices included in the hair analysis result with a preset reference range and may classify first indices based on whether the first index is included in the reference range. In an embodiment, the first index may be a ratio of one preset mineral of the nutritional minerals to another preset mineral thereof.

In addition, the service-providing apparatus 100 may classify the second indices based on whether each of the plural second indices included in the hair analysis result exceeds a preset safe level. In an embodiment, the second index may be a ratio of one preset nutritional mineral of the nutritional minerals to one preset harmful heavy metal of the harmful heavy metals.

In addition, the service-providing apparatus 100 may generate a nutritional mineral ratio test result based on the classification results of the first indices, and may provide the generated nutritional mineral ratio test result to the user terminal 200.

In addition, the service-providing apparatus 100 may generate nutritional mineral/harmful metal ratio test results based on the classification results of the second indices, and may provide the generated nutritional mineral/harmful metal ratio test results to the user terminal 200.

In addition, the service-providing apparatus 100 may calculate an intake necessity level corresponding to each of the nutritional minerals, may determine nutritional minerals requiring priority intake based on the intake necessity level, and may provide the determined nutritional minerals to the user terminal 200.

In addition, the service-providing apparatus 100 may calculate an intake caution level corresponding to each of the nutritional minerals, may determine nutritional minerals, whose intake should be restricted, based on the intake caution level, and may provide the determined nutritional minerals to the user terminal 200.

In addition, the service-providing apparatus 100 may provide a detailed survey to multiple risk factors, which may cause absorption of harmful heavy metals requiring attention, to the user terminal 200, may receive a detailed survey result, which includes the response to the detailed survey, from the user terminal 200, and may determine risk factors, which the user should be careful about, based on the detailed survey result.

In addition, the service-providing apparatus 100 may determine food suitable for the user's companion animal, and may provide information on the determined food to the user terminal 200.

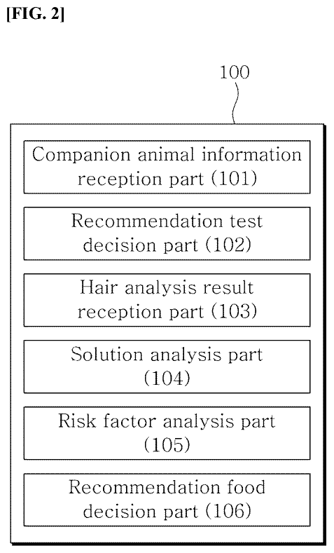

FIG. 2 is a block diagram exemplarily illustrating a functional module of the service-providing apparatus 100 according to FIG. 1.

Referring to FIG. 2, the service-providing apparatus 100 includes a companion animal information reception part 101. The companion animal information reception part 101 may receive the companion animal information about the user's companion animal from the user terminal 200. The service-providing apparatus 100 may provide the user interface capable of selecting or entering the companion animal information about the user's companion animal to the user terminal 200.

The companion animal information may include information on age, neutering, weight, personality, species, breed, body type, activity information, disease, staple diet, and snacks.

In an embodiment, the companion animal information reception part 101 may receive an age selected or inputted in a preset range. In an embodiment, the age of the companion animal may be set to 1 to 20 years old. However, the age is not limited thereto, and various ranges of ages may be set.

In an embodiment, the companion animal information reception part 101 may receive whether the companion animal is neutered. The user may select or input whether the companion animal has been neutered or not through the user interface.

In an embodiment, the companion animal information reception part 101 may receive information on the weight of the companion animal. In an embodiment, a weight range of the companion animal may be set to 1 to 80 Kg. However, the weight is not limited thereto, and various ranges of weights may be set. In addition, the units of the weight may be Kg, g and 1b.

In an embodiment, the companion animal information reception part 101 may receive information on a species selected from preset options. In the illustrated embodiment, the species of the companion animal may be a dog or a cat. However, the present invention is not limited thereto.

In an embodiment, the companion animal information reception part 101 may receive information on the breed of the companion animal selected from preset options. In an embodiment, the breed of the companion animal may be one selected from Samoyed, Jindodog, Bulldog and Pit Bull Terrier. However, the present invention is not limited thereto, and all breeds registered as dog breeds may be provided to the user as options.

In an embodiment, the companion animal information reception part 101 may receive information on the personality of the companion animal selected from preset options. In an embodiment, if the species is different, the options for the personality may be set differently.

In an embodiment, the companion animal information reception part 101 may receive information on the body shape of the companion animal selected from preset options. In an embodiment, if the species and breed are different, the options for body shape may be set differently.

In an embodiment, the companion animal information reception part 101 may receive information on the activity level of the companion animal selected from preset options. In an embodiment, if the species of the companion animal is a dog, active, normal, inactive, etc. may be set as options. In an embodiment, if the species is different, the options for the activity level may be set differently.

In an embodiment, the companion animal information reception part 101 may receive information on the disease of the companion animal selected from preset options. In an embodiment, if the species is different, the options for the disease may be set differently.

In an embodiment, the companion animal information reception part 101 may receive information on the staple diet of the companion animal selected from preset options. In an embodiment, brands and product names may be set as options for a staple diet.

In an embodiment, the companion animal information reception part 101 may receive information on the snack of the companion animal selected from preset options. In an embodiment, brands and product names may be set as options for a snack.

In addition, the companion animal information reception part 101 may provide a first survey including a plurality of questions about each of harmful heavy metals to the user terminal 200, and may receive a first survey result including a plurality of responses to the plural questions from the user terminal 200. In addition, the companion animal information reception part 101 may provide a second survey including a plurality of questions about each of the nutritional minerals to the user terminal 200, and may receive a second survey result including a plurality of responses to the plural questions from the user terminal 200. The companion animal information reception part 101 may provide the user interface for inputting responses to the first survey and the second survey to the user terminal 200. In an embodiment, mercury (Hg), arsenic (As), cadmium (Cd), lead (Pb), aluminum (Al), nickel (Ni), and uranium (U) may be considered as harmful heavy metals. In an embodiment, calcium (Ca), sodium (Na), potassium (K), phosphorus (P), magnesium (Mg), copper (Cu), zinc (Zn), iron (Fe), manganese (Mn), chromium (Cr), selenium (Se), and the like may be considered as nutritional minerals. In an embodiment, the first survey including a plurality of questions about each of harmful heavy metals may be stored in the database of the service-providing apparatus 100. In an embodiment, the second survey including a plurality of questions about each of the nutritional minerals may be stored in the database of the service-providing apparatus 100.

For example, whether a companion animal is exposed to secondhand smoke, whether a companion animal drinks groundwater, whether preservatives containing arsenic are used in wooden products, etc. may be set as questions about arsenic (As). For example, questions about aluminum (Al) may be set as whether the frequency of feeding wet food is above a preset standard, whether aluminum containers are used, and the like. For example, whether the concentration of fine dust is checked before going for a walk, whether a companion animal plays with coins, and the like may be set as questions about nickel (Ni).

For example, questions related to the intake of nutritional minerals may be set. Questions related to the intake of dairy products, anchovies, seaweed, and the like may be set as questions about calcium. Questions related to the intake of tofu, beans, hijikia, kelp, milk, mackerel, bananas, and the like may be set as questions about magnesium. Questions related to the intake of foods with a high salt content such as general diet of humans, and the like may be set as questions about sodium. Questions related to the intake of sweet potatoes, potatoes, tomatoes, cucumbers, pumpkins, apples, bananas, and the like may be set as questions about potassium. Questions related to the intake of beef liver, pork liver, legumes, mushrooms, and the like may be set as questions about copper. Questions related to the intake of protein foods such as red meat, chicken, cabbage, and the like may be set as questions about zinc. Questions related to the intake of meat, fish, eggs, milk, tofu, vegetables, and the like may be set as questions about phosphorus. Questions related to the intake of protein foods such as red meat, poultry, fish, and the like may be set as questions about iron. Questions related to the intake of chickpeas, lotus root, and the like may be set as questions about manganese. Questions related to the intake of cheese, meat, mushrooms, and the like may be set as questions about chromium. Questions related to the intake of meat internal organs, fish (mackerel), meat (beef, turkey, chicken), broccoli, eggs, and the like may be set as questions about selenium.

Referring to FIG. 2 again, the service-providing apparatus 100 includes a recommendation test decision part 102.

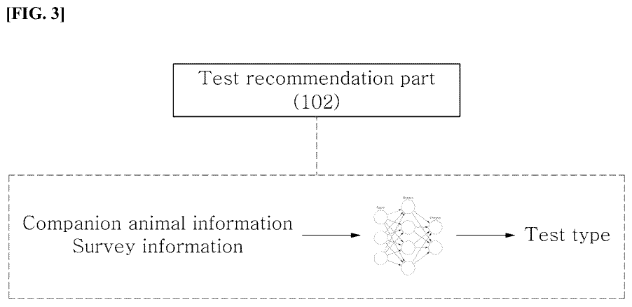

FIG. 3 is a diagram conceptually illustrating a process of determining the type of test, recommended to the user's companion animal, by the recommendation test decision part 102 according to FIG. 2.

The recommendation test decision part 102 may input the age, neutering status, weight, personality, species, breed, body shape, activity information, disease, staple diet and snack included in the companion animal information and the first survey result as input values to a first machine-learning model that has been previously learned, and may obtain the possibility of an abnormality, related to harmful heavy metals, from the first machine-learning model.

The first machine-learning model may be machine-learned to output the possibility of an abnormality related to harmful heavy metals when receiving the age, neutering status, weight, personality, species, breed, body shape, activity information, disease, staple diet and snack included in the companion animal information and the first survey result as input values. For the learning of the first machine-learning model, learning data generated by labeling the age, neutering status, weight, personality, species, breed, body shape, activity information, disease, staple diet and snack included in the companion animal information and the first survey result with the possibility of an abnormality related to harmful heavy metals may be used. In an embodiment, For the learning of the first machine-learning model, random forest, Xgboost, multiple regression analysis, and the like may be used.

The recommendation test decision part 102 may input the age, neutering status, weight, personality, species, breed, body shape, activity information, disease, staple diet and snack included in the companion animal information and the second survey result as input values to a second machine-learning model that has been previously learned, and may obtain the possibility of an abnormality related to nutritional minerals from the second machine-learning model.

The second machine-learning model may be machine-learned to output the possibility of an abnormality related to nutritional minerals when receiving the age, neutering status, weight, personality, species, breed, body shape, activity information, disease, staple diet and snack included in the companion animal information and the second survey result as input values. For the learning of the second machine-learning model, learning data generated by labeling the age, neutering status, weight, personality, species, breed, body shape, activity information, disease, staple diet and snack included in the companion animal information and the second survey result with the possibility of an abnormality related to nutritional minerals may be used. In an embodiment, For the learning of the second machine-learning model, random forest, Xgboost, multiple regression analysis, and the like may be used.

In addition, when the possibility of an abnormality related to harmful heavy metals obtained from the first machine-learning model is greater than a preset standard possibility, and the possibility of an abnormality related to nutritional minerals obtained from the second machine-learning model is equal to or lower than a preset standard possibility, the recommendation test decision part 102 may determine a first test, which performs tests only related to harmful heavy metals, as a recommended test to be provided to the user.

In addition, when the possibility of an abnormality related to harmful heavy metals obtained from the first machine-learning model is equal to or lower than the preset standard possibility, and the possibility of an abnormality related to nutritional minerals obtained from the second machine-learning model is greater than the preset standard possibility, the recommendation test decision part 102 may determine a second test, which performs tests only related to nutritional minerals, as a recommended test to be provided to the user.

In addition, when the possibility of an abnormality related to harmful heavy metals obtained from the first machine-learning model is greater than the preset standard possibility, and the possibility of an abnormality related to nutritional minerals obtained from the second machine-learning model is greater than the preset standard possibility, the recommendation test decision part 102 may determine a third test, which performs all tests related to harmful heavy metals and nutritional minerals, as a recommended test to be provided to the user.

In addition, the recommendation test decision part 102 may provide the determined recommended test to the user terminal 200.

Referring to FIG. 2 again, the service-providing apparatus 100 includes a hair analysis result reception part 103.

The hair analysis result reception part 103 may receive a hair analysis result, which is a result of analyzing the hair of the user's companion animal, through the input interface device. In addition, the hair analysis result reception part 103 may receive a hair analysis result, which is a result of analyzing the hair of the user's companion animal, from a separate server.

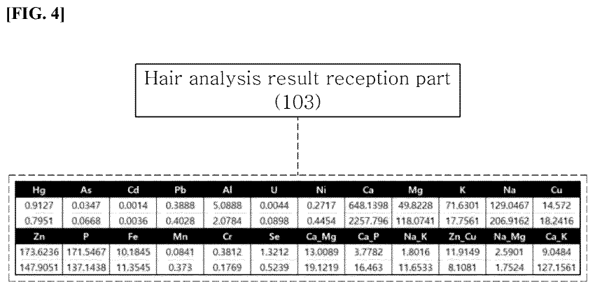

FIG. 4 is a diagram conceptually illustrating a process of receiving a hair analysis result by the hair analysis result reception part 103 according to FIG. 2.

The hair analysis result may include the heavy metal level of each of the harmful heavy metals included in the hair. In addition, the hair analysis result may include the mineral level of each of the nutritional minerals included in hair. In an embodiment, the units of the heavy metal level and the mineral level may be μg/g.

The hair analysis result may include a first index which is a ratio of one preset nutritional mineral of nutritional minerals included in hair to another preset nutritional mineral thereof. In the illustrated embodiment, a ratio of the mineral level of magnesium to the mineral level of calcium, a ratio of the mineral level of phosphorus to the mineral level of calcium, a ratio of the mineral level of potassium to the mineral level of sodium, a ratio of the mineral level of copper to the mineral level of zinc, a ratio of the mineral level of magnesium to the mineral level of sodium and a ratio of the mineral level of potassium to the mineral level of calcium may be set as first indices.

The hair analysis result may include a ratio of one preset nutritional mineral of nutritional minerals included in hair to one preset harmful heavy metal of the harmful heavy metals as a second index. In an embodiment, a ratio of the heavy metal level of mercury to the mineral level of zinc, a ratio of the heavy metal level of mercury to the mineral level of selenium, a ratio of the heavy metal level of lead to the mineral level of calcium, a ratio of the heavy metal level of lead to the heavy metal level of iron, a ratio of the heavy metal level of lead to the mineral level of zinc and a ratio of the heavy metal level of cadmium to the mineral level of zinc may be set as second indices.

In an embodiment, a hair analysis result may be obtained through a process of preparing a sample solution by containing the hair of a companion animal in a nitric acid solution, pretreating the prepared sample solution through heating, and mass-analyzing the pretreated sample solution using inductively coupled plasma mass spectrometry (ICP-MS). However, the present invention is not limited thereto, and known various methods of obtaining harmful heavy metals and nutritional minerals contained in hair from hair may be used.

Referring to FIG. 2 again, the service-providing apparatus 100 includes a solution analysis part 104.

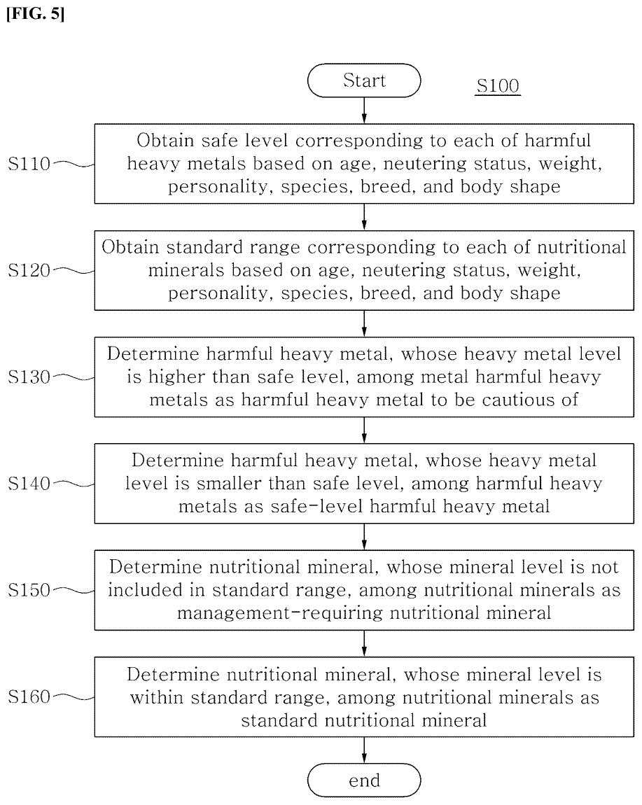

FIG. 5 is a flowchart illustrating a process of determining harmful heavy metals requiring caution among harmful heavy metals and nutritional minerals requiring management among nutritional minerals by a solution analysis part 104 according to FIG. 2.

First, the solution analysis part 104 obtains the safe level corresponding to each of harmful heavy metals based on the age, neutering status, weight, personality, species, breed, and body shape (S110).

The solution analysis 101 may input the age, neutering status, weight, personality species, breed and body shape included in the companion animal information as input values to a third machine-learning model that has been previously learned, and may obtain the safe level of each of harmful heavy metals from the third machine-learning model.

The third machine-learning model may be machine-learned to output the safe level of each of harmful heavy metals when receiving the age, neutering status, weight, personality, species, breed and body shape included in the companion animal information as input values. For the learning of the third machine-learning model, learning data generated by labeling the age, neutering status, weight, personality, species, breed and body shape included in the companion animal information with the safe level of each of harmful heavy metals may be used. In an embodiment, for the learning of the third machine-learning model, random forest, Xgboost, multiple regression analysis, etc. may be used. For example, the safe levels of mercury, arsenic, cadmium, lead, aluminum, nickel and uranium may be obtained as 0.50, 0.20, 0, 100, 1.0, 30, 1.50 and 0.100, respectively.

There is a generally set safe level for each harmful heavy metal, but there may be slight differences for each companion animal. To conduct an accurate examination of a companion animal, a safe level suitable for the companion animal may be obtained based on the characteristics of the companion animal, and the health status of the companion animal may be determined based on the obtained safe level.

In addition, the solution analysis part 104 obtains a standard range corresponding to each of the nutritional minerals based on the age, neutering status, weight, personality, species, breed, and body shape (S120).

The solution analysis is part 104 may input the age, neutering status, weight, personality, species, breed and body shape included in the companion animal information as input values to a fourth machine-learning model that has been previously learned, and may obtain a standard range of each of the nutritional minerals from the fourth machine-learning model.

The fourth machine-learning model may be machine-learned to output the standard range of each of the nutritional minerals when receiving the age, neutering status, weight, personality, species, breed and body shape included in the companion animal information as input values. For the learning of the fourth machine-learning model, learning data generated by labeling the age, neutering status, weight, personality, species, breed and body shape included in the companion animal information with the standard range of each of the nutritional minerals may be used. In an embodiment, for the learning of the fourth machine-learning model, random forest, Xgboost, multiple regression analysis, etc. may be used. For example, the standard ranges for calcium, magnesium, sodium, potassium, copper, zinc, phosphorus, iron, manganese, chromium, and selenium may be obtained as 200 to 1400, 25 to 220, 200 to 2000, 70 to 600, 7.0 to 25.0, 140 to 240, 200 to 400, 14 to 100, 0.25 to 3.00, 0.20 to 1.00 and 0.70 to 1.80, respectively.

The standard range for each of the nutritional minerals is generally set, but there may be slight differences for each companion animal. To conduct an accurate examination of a companion animal, a standard range suitable for the companion animal may be obtained based on the characteristics of the companion animal, and the health status of the companion animal may be determined based on the obtained standard range.

In addition, the solution analysis part 104 determines a harmful heavy metal, whose heavy metal level is higher than the safe level, among the harmful heavy metals as a harmful heavy metal to be cautious of (S130), and determines a harmful heavy metal, whose heavy metal level is lower than the safe level, among the harmful heavy metals as a safe-level harmful heavy metal (S140).

For example, when the safe level of mercury is 0.50 and the heavy metal level of mercury is 0.04, the heavy metal level of mercury is smaller than the safe level, so that the solution analysis part 104 may determine mercury as a safe-level harmful heavy metal. When the safe level of lead is 1.0, and the heavy metal level of lead is 2.5, the heavy metal level of lead is higher than the safe level, the solution analysis part 104 may determine lead as a harmful heavy metal to be cautious of.

In addition, the solution analysis part 104 determines a nutritional mineral, whose mineral level is not included in the standard range, among nutritional minerals as a management-requiring nutritional mineral (S150), and determines a nutritional mineral, whose mineral level is within the standard range, among nutritional minerals as a standard nutritional mineral (S160).

For example, when the standard range of calcium is 200 to 1400 and the mineral level of calcium is 172, the mineral level of calcium is not included in the standard range, so that the solution analysis part 104 may determine calcium as a management-requiring nutritional mineral. When the standard range of sodium is 200 to 2000 and the mineral level of sodium is 710, the mineral level of sodium is included in the standard range, so that the solution analysis part 104 may determine sodium as a standard nutritional mineral.

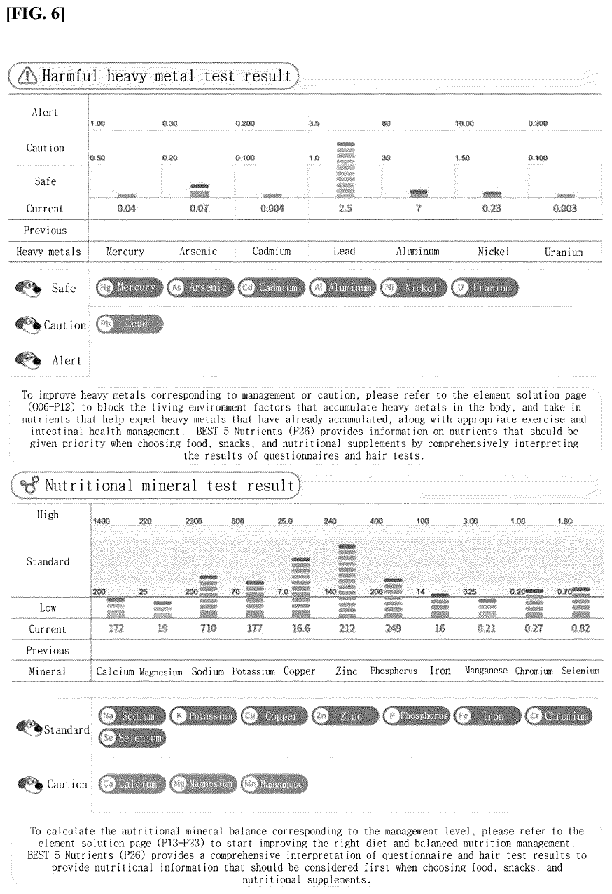

FIG. 6 is a diagram conceptually illustrating a process where the solution analysis part 104 according to FIG. 2 provides a user with a test result of harmful heavy metals and a test result of nutritional minerals.

In the test result of harmful heavy metals, the harmful heavy metals may be classified into harmful heavy metals determined as harmful heavy metals to be cautious of; and harmful heavy metals determined as safe-level harmful heavy metals. In addition, the harmful heavy metals determined as harmful heavy metals to be cautious of; and the harmful heavy metals determined as safe-level harmful heavy metals may be displayed in different colors.

The test result of harmful heavy metals may include the safe level and heavy metal level of each of harmful heavy metals.

In the test result of nutritional minerals, the nutritional minerals may be classified into nutritional minerals determined as management-requiring nutritional minerals; and nutritional minerals determined as standard nutritional minerals. In addition, the nutritional minerals determined as management-requiring nutritional minerals and the nutritional minerals determined as standard nutritional minerals may be displayed in different colors.

The test result of nutritional minerals may include the standard range and mineral level of each of the nutritional minerals.

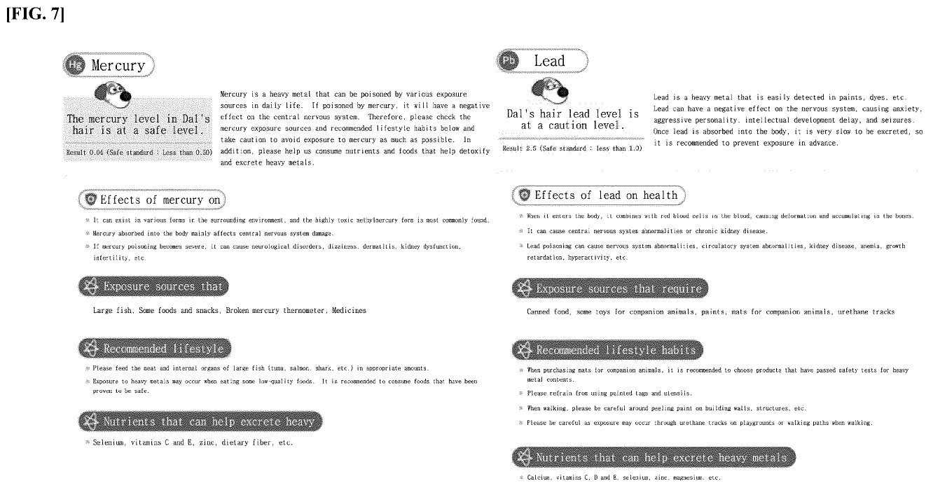

FIG. 7 is a diagram conceptually illustrating a process where the solution analysis part 104 according to FIG. 2 provides a test result of harmful heavy metals to a user.

In the database of the service-providing apparatus 100, descriptions of harmful heavy metals and harmful heavy metals, effects of harmful heavy metals on health, exposure sources of harmful heavy metals, lifestyle habits that can prevent absorption of harmful heavy metals and nutrients that help excrete harmful heavy metals are matched and stored in advance.

The test result of harmful heavy metals may include harmful heavy metals and a description of each thereof, effects of harmful heavy metals on health, exposure sources of harmful heavy metals, lifestyle habits that can prevent absorption of harmful heavy metals, and nutrients that help excrete harmful heavy metals. In addition, the test result may include the safe level and heavy metal level corresponding to each of harmful heavy metals.

In the illustrated embodiment, the test result of harmful heavy metals includes a safe level and heavy metal level corresponding to mercury, a description of mercury, the effects of mercury on health, exposure sources of mercury, lifestyle habits that can prevent absorption of mercury, and nutrients that help excrete mercury. In addition, since the heavy metal level of mercury is smaller than the safe level, an indication that mercury is a safe-level harmful heavy metal is included.

In the illustrated embodiment, the test result of harmful heavy metals includes a safe level and heavy metal level corresponding to lead, a description of lead, effects of lead on health, exposure sources of lead, lifestyle habits that can prevent absorption of lead, and nutrients that help excrete lead. In addition, since the heavy metal level of lead is higher than the safe level, an indication that lead is a harmful heavy metal to be cautious of is included.

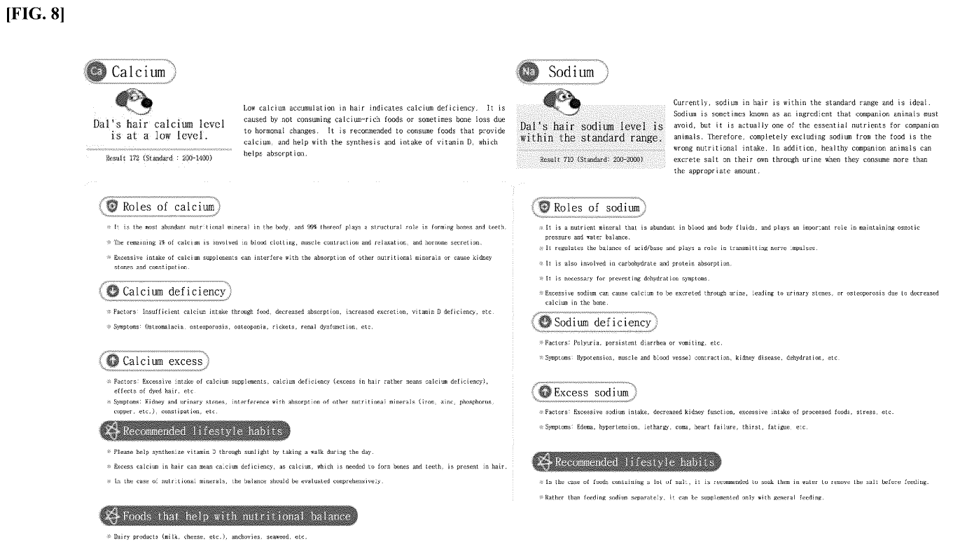

FIG. 8 is a diagram conceptually illustrating a process where the solution analysis part 104 according to FIG. 2 provides a user with a test result of nutritional minerals.

In the database of the service-providing apparatus 100, nutritional minerals and descriptions thereof, effects of nutritional minerals on health, causes of nutritional mineral deficiency, symptoms occurring when nutritional minerals are deficient, causes of nutritional mineral excess, symptoms occurring when nutritional minerals are excessive, lifestyle habits that can maintain appropriate levels of nutritional minerals, and nutrients that help maintain appropriate levels of nutritional minerals are matched and stored in advance.

In the test result of nutritional minerals, a description of a nutritional mineral corresponding to each of harmful heavy metals, effects of nutritional minerals on health, causes of nutritional mineral deficiency, symptoms occurring when nutritional minerals are deficient, causes of nutritional mineral excess, symptoms occurring when nutritional minerals are excessive, lifestyle habits that can maintain appropriate levels of nutritional minerals, and nutrients that help maintain appropriate levels of nutritional minerals may be included. In addition, the test result may include a standard range and mineral level corresponding to each of the nutritional minerals.

In the illustrated embodiment, the test result of nutritional minerals includes a standard range and mineral level corresponding to calcium, a description of calcium, effects of calcium on health, causes of calcium deficiency, symptoms that occur when calcium is deficient, causes of calcium excess, symptoms that occur when calcium is excessive, lifestyle habits that can maintain an appropriate level of calcium, and nutrients that help maintain an appropriate level of calcium are included. In addition, since the mineral level of calcium is not included in the standard range, an indication that calcium is a nutritional mineral of interest is included.

In the illustrated embodiment, the test result of nutritional minerals includes a standard range and mineral level corresponding to sodium, a description of sodium, effects of sodium on health, causes of sodium deficiency, symptoms occurring when sodium is deficient, causes of sodium excess, symptoms occurring when sodium is excessive, lifestyle habits that can maintain an appropriate level of sodium, and nutrients that help maintain an appropriate level of sodium are included. In addition, since the mineral level of sodium is included in the standard range, an indication that sodium is a standard nutritional mineral is included.

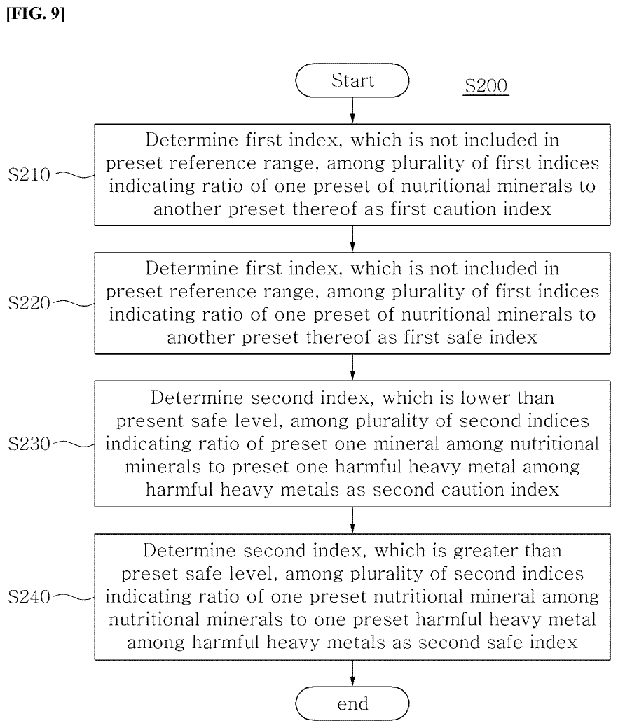

FIG. 9 is a flowchart illustrating a process of determining first and second indices, which require attention, among the first indices and second indices by the solution analysis part 104 according to FIG. 2.

The solution analysis part 104 determines a first index, which is not included in a preset reference range, among the first indices as a first caution index (S210), and determines a first index, which is not included in the preset reference range, among the first indices as a first safety index (S220).

In the database of the service-providing apparatus 100, each of the first indices and the reference range may be matched and stored in advance. In an embodiment, reference ranges for a ratio of the mineral level of magnesium to the mineral level of calcium, a ratio of the mineral level of phosphorus to the mineral level of calcium, a ratio of the mineral level of potassium to the mineral level of sodium, a ratio of the mineral level of copper to the mineral level of zinc, a ratio of the mineral level of magnesium to the mineral level of sodium and a ratio of the mineral level of potassium to the mineral level of calcium may be set to 1.5 to 18.4, 1.2 to 6.8, 0.6 to 20.5, 7.1 to 28.6, 11.3 to 29.3 and 0.7 to 12.9.

In addition, the solution analysis part 104 determines a second index, which is lower than the present safe level, among the second indices as a second caution index (S230), and determines a second index, which is higher than the preset safe level, among the second indices as a second safety index (S240).

In the database of the service-providing apparatus 100, each of the second indices and the safe level may be matched and stored in advance. In an embodiment, safe levels for a ratio of the heavy metal level of mercury to the mineral level of zinc, a ratio of the heavy metal level of mercury to the mineral level of selenium, a ratio of the heavy metal level of lead to the mineral level of calcium, a ratio of the heavy metal level of lead to the heavy metal level of iron, a ratio of the heavy metal level of lead to the mineral level of zinc and a ratio of the heavy metal level of cadmium to the mineral level of zinc may be set to 300, 0.6, 67.6, 205, 13.6 and 600.

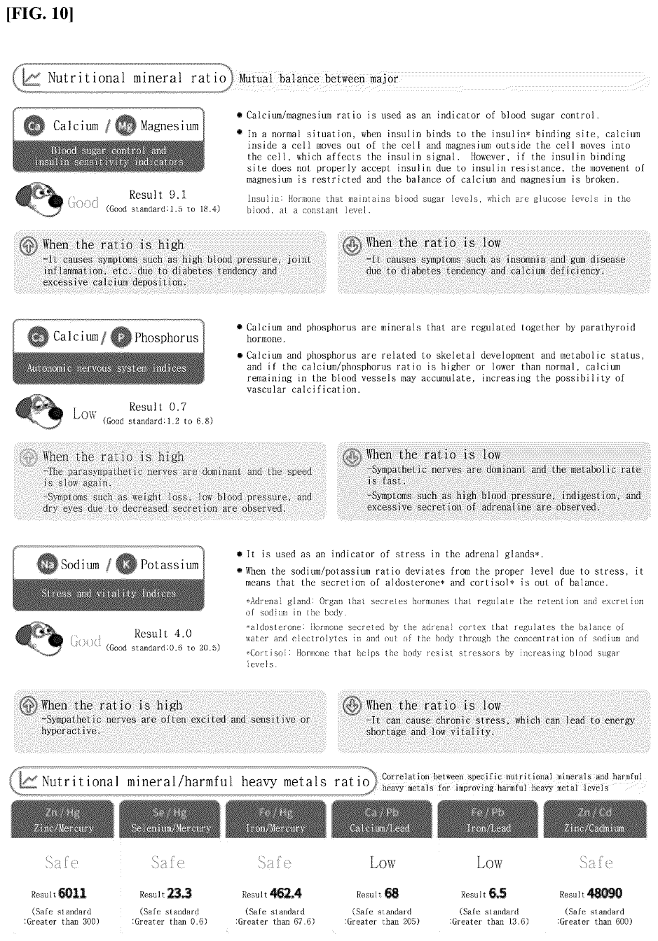

FIG. 10 is a diagram conceptually illustrating a process where test results of the first indices and second indices are provided to a user by the solution analysis part according to FIG. 2.

In the database of the service-providing apparatus 100, the first indices and a description of each thereof, symptoms when the first index is greater than the maximum value of the reference range, and symptoms when the first index is smaller than the minimum value of the reference range are matched and stored.

The test result of the first indices may include the first indices and a description corresponding to each thereof, symptoms when the first index is greater than the maximum value of the reference range, and symptoms when the first index is smaller than the minimum value of the reference range.

In the illustrated embodiment, the test result of the first indices includes descriptions of a ratio of the mineral level of magnesium to the mineral level of calcium, and a ratio of the mineral level of magnesium to the mineral level of calcium, symptoms when a ratio of the mineral level of magnesium to the mineral level of calcium is greater than the maximum value of the reference range, and symptoms when a ratio of the mineral level of magnesium to the mineral level of calcium is smaller than the minimum value of the reference range. In addition, since the ratio, i.e., 9.1, of the mineral level of magnesium to the mineral level of calcium in the test result of the first indices is included in a reference range of 1.5 to 18.4, an indication that the ratio of the mineral level of magnesium to the mineral level of calcium is the first safety index is included.

In the illustrated embodiment, the test result of the second indices includes a ratio of the heavy metal level of mercury to the mineral level of zinc and a safe level. In addition, since the ratio, i.e., 6011, of the heavy metal level of mercury to the mineral level of zinc in the test result of the second indices is greater than a safe level of 300, an indication that the ratio of the heavy metal level of mercury to the mineral level of zinc is the second safety index is included.

In the illustrated embodiment, the test result of the second indices includes a ratio of the heavy metal level of lead to the mineral level of calcium and a safe level. In addition, since the ratio, i.e., 68, of the heavy metal level of lead to the mineral level of calcium in the test result of the second indices is smaller than a safe level of 205, an indication that the ratio of the heavy metal level of lead to the mineral level of calcium is the second caution index is included.

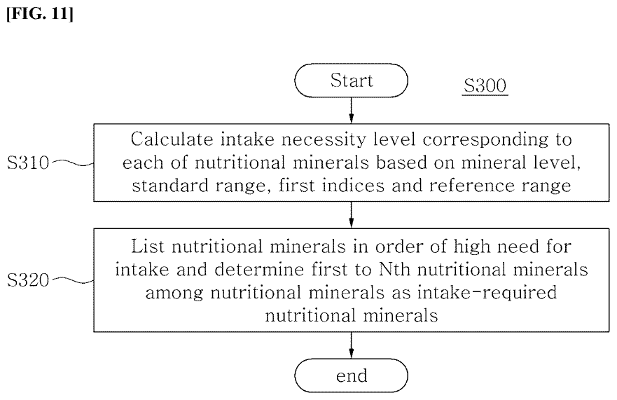

FIG. 11 is a flowchart illustrating a process of determining intake-required nutritional minerals by the solution analysis part 104 according to FIG. 2.

The solution analysis part 104 calculates an intake necessity level corresponding to each of the nutritional minerals based on the mineral level, the standard range, the first indices and the reference range (S310).

In an embodiment, the solution analysis part 104 may determine a value, obtained by multiplying the standard value of the nutritional mineral by the weighted value, as an intake necessity level.

When the mineral level is smaller than the minimum value of the standard range, the solution analysis part 104 may determine a value, which is obtained by adding a correction value to a value that is obtained by dividing a first difference value between the minimum value of the standard range and the mineral level by a second difference value between the maximum value of the standard range and the minimum value, as a standard value.

When the mineral level is smaller than the median value of the standard range and greater than the minimum value thereof, the solution analysis part 104 may determine a value, obtained by dividing a third difference value between the median value of the standard range and the mineral level by the second difference value between the maximum value of the standard range and the minimum value thereof, as a standard value. In an embodiment, a correction value is set to be greater than the maximum value of a value that can be derived by dividing the third difference value by the second difference value.

When the mineral level is greater than the median value of the standard range, the solution analysis part 104 may determine the minimum value of a value that can be derived by dividing the third difference value by the second difference value as a standard value.

When the first index is smaller than the minimum value of the reference range, the solution analysis part 104 may assign a weighted value of greater than 1 to a nutritional mineral corresponding to the numerator of the first index, and may assign a weighted value of 1 to a nutritional mineral corresponding to the denominator thereof.

When the first index is greater than the maximum value of the reference range, the solution analysis part 104 may assign a weighted value of greater than 1 to a nutritional mineral corresponding to the denominator of the first index, and may assign a weighted value of 1 to a nutritional mineral corresponding to the numerator thereof.

When the first index is included in the reference range, the solution analysis part 104 may assign a weighted value of 1 to nutritional minerals corresponding to the denominator and numerator of the first index.

In addition, the solution analysis part 104 lists nutritional minerals in order of high need for intake, and determines the first to Nth (where N is a preset natural number greater than 1 and less than or equal to the number of nutritional minerals) nutritional minerals among nutritional minerals as intake-required nutritional minerals (S320).

In an embodiment, when N is 5, the solution analysis part 104 may determine five nutritional minerals as intake-required nutritional minerals in order of high need for intake.

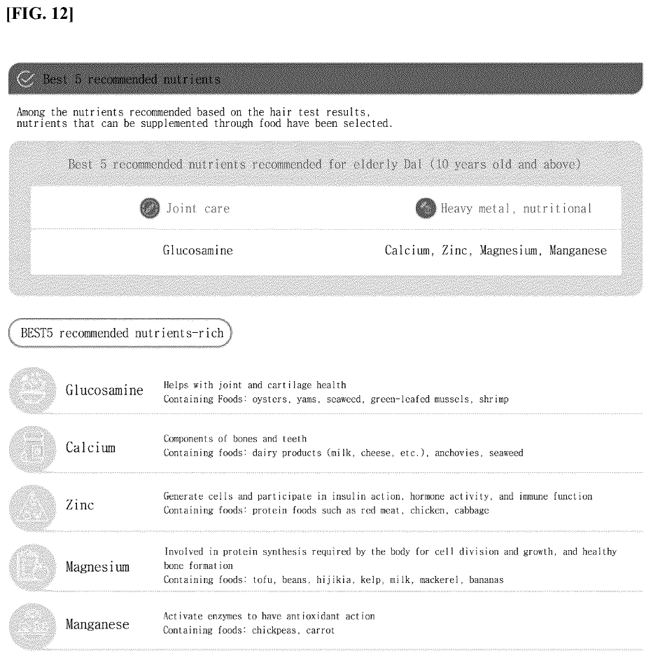

FIG. 12 is a diagram conceptually illustrating a process of providing intake-required nutritional minerals and foods containing the intake-required nutritional minerals to a user by the solution analysis part 104 according to FIG. 2.

In the database of the service-providing apparatus 100, each of the nutritional minerals, the role of the nutritional mineral, and food containing the nutritional mineral are matched and stored.

The solution analysis part 104 may search nutritional mineral's roles previously matched with nutritional minerals determined as intake-required nutritional minerals, and foods containing the nutritional mineral, and may provide information on the searched nutritional mineral′ roles and nutritional mineral-containing foods to the user terminal 200.

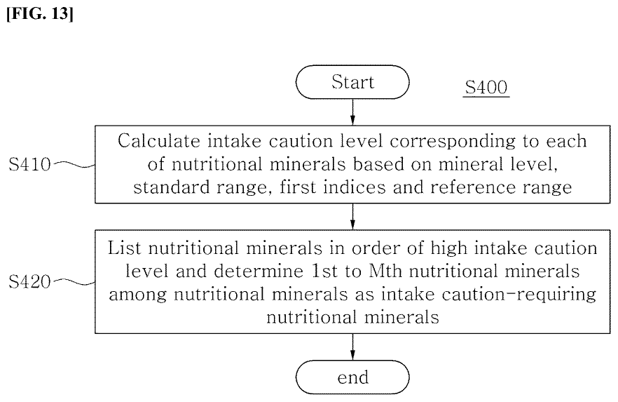

FIG. 13 is a flowchart illustrating a process of determining intake caution-requiring nutritional minerals by the solution analysis part 104 according to FIG. 2.

The solution analysis part 104 calculates an intake caution level corresponding to each of the nutritional minerals based on the mineral level, the standard range, the first indices and the reference range (S410).

In an embodiment, the solution analysis part 104 may determine a value, obtained by multiplying the standard value of the nutritional mineral by a weighted value, an intake caution level.

When the mineral level is greater than the maximum value of the standard range, the solution analysis part 104 may determine a value, obtained by adding a correction value to a value obtained by dividing a first difference value between the maximum value of the standard range and the mineral level by a second difference value between the maximum value of the standard range and the minimum value thereof, as a standard value.

When the mineral level is greater than the median value of the standard range and greater than the maximum value, the solution analysis part 104 may determine a value, obtained by dividing a third difference value between the median value of the standard range and the mineral level by the second difference value between the maximum value of the standard range and the minimum value thereof, as a standard value. In an embodiment, a correction value is set to be greater than the maximum value of a value that can be derived by dividing the third difference value by the second difference value.

When the mineral level is smaller than the median value of the standard range, the solution analysis part 104 may determine the minimum value of a value, which can be derived by dividing the third difference value by the second difference value, as a standard value.

When the first index is smaller than the minimum value of the reference range, the solution analysis part 104 may assign a weighted value of greater than 1 to a nutritional mineral corresponding to the denominator of the first index, and may assign a weighted value of 1 to a nutritional mineral corresponding to the numerator of the first index.

When the first index is greater than the maximum value of the reference range, the solution analysis part 104 may assign a weighted value of greater than 1 to a nutritional mineral corresponding to the numerator of the first index, and may assign a weighted value of 1 to a nutritional mineral corresponding to the denominator thereof.

When the first index is included in the reference range, the solution analysis part 104 may assign a weighted value of 1 to nutritional minerals corresponding to the denominator and numerator of the first index.

In addition, the solution analysis part 104 lists nutritional minerals in order of high intake caution level, and determines the 1st to Mth (where M is a preset natural number greater than 1 and less than or equal to the number of nutritional minerals) nutritional minerals among the nutritional minerals as intake caution-requiring nutritional minerals (S420).

In an embodiment, when M is 3, the solution analysis part 104 may determine three nutritional minerals as intake caution-requiring nutritional minerals in order of high intake caution level.

In the database of the service-providing apparatus 100, nutritional minerals and the roles of each thereof, and foods containing the nutritional mineral are matched and stored.

The solution analysis part 104 may search nutritional minerals determined as intake caution-requiring nutritional minerals, previously matched nutritional mineral's roles and foods containing the nutritional minerals, and may provide information on the searched nutritional mineral′ roles and nutritional mineral-containing foods to the user terminal 200.

Referring to FIG. 2 again, the service-providing apparatus 100 includes a risk factor analysis part 105.

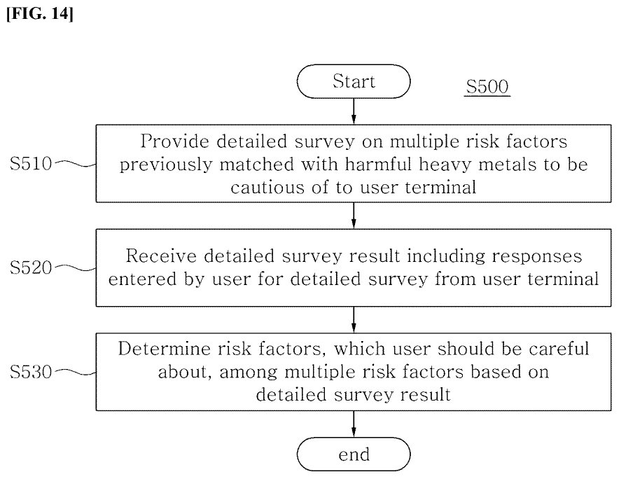

FIG. 14 is a flowchart illustrating a process of determining risk factors, which a user should be careful about, by the risk factor analysis part 105 according to FIG. 2.

When the solution analysis part 104 determines that a harmful heavy metal to be cautious of among harmful heavy metals exists, the risk factor analysis part 105 may provide a detailed survey, which contains a plurality of questions about multiple risk factors that may cause absorption of the harmful heavy metal to be cautious of, to the user terminal 200, and may receive a detailed survey result containing a response to the detailed survey from the user terminal 200.

The risk factor analysis part 105 provides the detailed survey for the multiple risk factors, previously matched with the harmful heavy metal to be cautious of, to a user terminal (S510).

In the database of the service-providing apparatus 100, harmful heavy metals and a detailed survey for multiple risk factors of each of the harmful heavy metals are matched and stored. In an embodiment, risk factors refer to situations in which a companion animal may be exposed to cadmium during its life. For example, a detailed survey for cadmium may contain questions about whether a companion animal is exposed to secondhand smoke, whether paint is used on toys for the companion animal, whether seafood is fed in addition to food or snacks, etc.

In addition, the risk factor analysis part 105 receives the detailed survey result containing the responses, entered by the user, to the detailed survey, from the user terminal 200 (S520).

In addition, the risk factor analysis part 105 determines risk factors, which the user should be careful about, among the multiple risk factors based on the detailed survey result (S530).

The risk factor analysis part 105 may determine risk factors, which the user should be careful about, based on the responses to the plural questions contained in the detailed survey result. For example, when the user responds that he/she feeds fish and shellfish in addition to food or snacks, feeding fish and shellfish in addition to food or snacks may be determined as a risk factor.

The risk factor analysis part 105 may provide the determined risk factors to the user terminal 200.

Referring to FIG. 2 again, the service-providing apparatus 100 includes a recommendation food decision part 106.

In the database, each of a plurality of foods, each of a plurality of nutritional components, and a component ratio of each of the nutritional components are matched and stored in advance. For example, food A and nutritional components contained in food A may be matched, and component ratios between food A and each nutrient may be matched and stored.

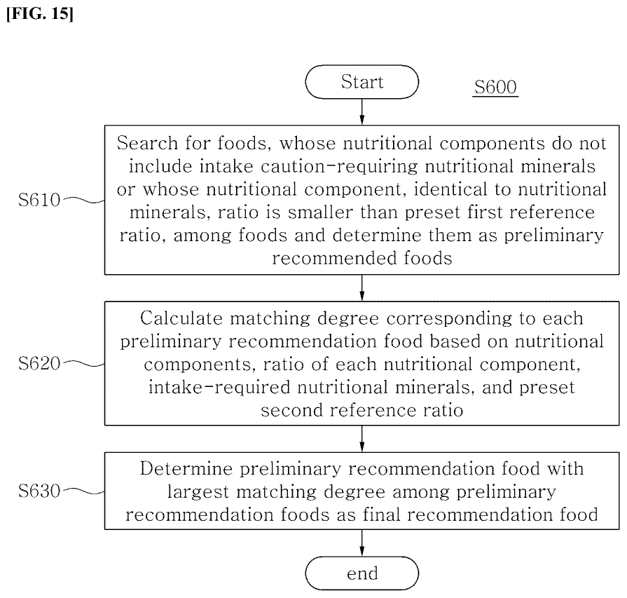

FIG. 15 is a flowchart illustrating a process of determining recommendation food by the recommendation food decision part 106 according to FIG. 2.

The recommendation food decision part 106 searches for foods in the nutritional components of which intake caution-requiring nutritional minerals are not contained, or foods in which a ratio of the same nutritional components as the nutritional minerals is smaller than a preset first reference ratio, and determined the searched foods as preliminary recommendation foods (S610).

The recommendation food decision part 106 searches for foods, which do not contain the intake caution-requiring nutritional minerals as nutritional components or in which a component ratio of the same nutritional minerals as the intake caution-requiring nutritional minerals is smaller than the preset first reference ratio, from the database. In an embodiment, the first reference ratio may be a ratio that can reduce the mineral levels of the intake caution-requiring nutritional minerals. In an embodiment, the first reference ratio may be set differently for each of the nutritional minerals.

In addition, the recommendation food decision part 106 calculates a matching degree corresponding to each preliminary recommendation food based on nutritional components, a ratio of each of nutritional components, intake-required nutritional minerals and a preset second reference ratio (S620).

The recommendation food decision part 106 determines nutritional components, which are identical to the intake-required nutritional mineral and have a component ratio higher than the preset second reference ratio, among the nutritional components included in the preliminary recommendation food as reference nutritional components for calculating a matching degree. In addition, the recommendation food decision part 106 may determine the sum of the intake necessity levels of intake-required nutritional minerals identical to the reference nutritional components as a matching degree of a preliminary recommendation food. In an embodiment, when there is only one reference nutritional component, the recommendation food decision part 106 may determine the intake necessity level of an intake-required nutritional mineral identical to the reference nutritional component as a matching degree of a preliminary recommendation food.

In addition, the recommendation food decision part 106 may determine a preliminary recommendation food with the highest matching degree among preliminary recommendation foods as a final recommendation food (S630).

The recommendation food decision part 106 may provide information on the final recommendation food to the user terminal 200.

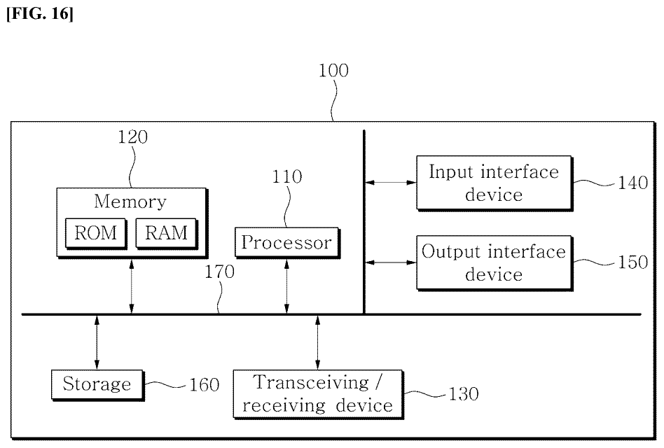

FIG. 16 exemplarily illustrates a hardware configuration of the service-providing apparatus 100 according to FIG. 1.

Referring to FIG. 16, the service-providing apparatus 100 may include at least one processor 110; and a memory that stores instructions that instruct the processor 110 to perform at least one operation.

The at least one operation may include the components 101 to 106 of the service-providing apparatus 100 described above or other functions or operation methods.

Here, the at least one processor 110 may mean a central processing unit (CPU), a graphics processing unit (GPU), or a dedicated processor on which methods according to embodiments of the present invention are performed. Each memory 120 and each storage 160 may be composed of at least one of a volatile storage medium and a nonvolatile storage medium.

For example, the memory 120 may be one of read-only memory (ROM) and random access memory (RAM), and the storage 160 may be a flash memory, a hard disk drive (HDD), a solid-state drive (SSD), various memory cards (for example, a micro SD card), etc.

In addition, the apparatus 100 may include a transceiver 130 that performs communication via a wireless network. In addition, the apparatus 100 may further include an input interface device 140, an output interface device 150, a storage 160, etc. Respective components included in the apparatus 100 may be connected to each other by a bus 170 and may communicate with each other.

Examples of the apparatus 100 include a communicable desktop computer, a laptop computer, a notebook, a smartphone, a tablet PC, a mobile phone, a smartwatch, a smart glass, an e-book reader, a portable multimedia player (PMP), a portable game console, a navigation device, a digital camera, a digital multimedia broadcasting (DMB) player, a digital audio recorder, a digital audio player, a digital video recorder, a digital video player, a personal digital assistant (PDA), and the like.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software.

Examples of a computer-readable recording medium may include hardware devices such as ROMs, RAMs and flash memories, which are specially configured to store and execute program commands. Examples of the program commands may include machine language code created by a compiler and high-level language code executable by a computer using an interpreter and the like. The hardware devices described above may be configured to operate as at least one software module to perform the operations of the invention, and vice versa.

In addition, the above-described method or apparatus may be implemented by combining all or part of constructions or functions thereof, or the constructions or functions may be separately implemented.

Although the present invention has been described above with reference to the embodiments of the present invention, those skilled in the art may variously modify and change the present invention without departing from the spirit and scope of the present invention as set forth in the claims below.

1. An apparatus for providing a total healthcare solution service through companion animal hair analysis, the apparatus comprising:
a hair analysis result reception part configured to receive a hair analysis result that comprises a mineral level of each of a plurality of nutritional minerals comprised in hair of a user's companion animal and a heavy metal level of each of a plurality of harmful heavy metals comprised in the hair; and
a solution analysis part configured to compare the mineral level of each of the nutritional minerals with a standard range corresponding to each of the nutritional minerals so as to classify the nutritional mineral into a management-requiring nutritional mineral or a standard nutritional mineral, and to compare the heavy metal level of each of the harmful heavy metals with the safe level corresponding to each of the harmful heavy metals so as to classify the harmful heavy metals into a harmful heavy metal to be cautious of or a safe-level harmful heavy metal.

2. The apparatus according to claim 1, wherein the solution analysis part:
determines a nutritional mineral, whose mineral level is not comprised in the standard range, among the nutritional minerals as the management-requiring nutritional mineral, determines a nutritional mineral, whose mineral level is comprised in the standard range, among the nutritional minerals as the standard nutritional mineral,
determines a harmful heavy metal, whose heavy metal level is equal to or higher than the safe level, among the harmful heavy metals as the harmful heavy metal to be cautious of, and
determines a harmful heavy metal, whose heavy metal level is smaller than the safe level, among the harmful heavy metals as the safe-level harmful heavy metal.

3. The apparatus according to claim 1, further comprising:
a companion animal information reception part configured to receive companion animal information comprising information on age, neutering, weight, personality, species, breed, body type, activity information, disease, staple diet, and snacks of the companion animal from a terminal of the user, receive a first survey result comprising a response to a first survey that comprises a plurality of questions about each of the harmful heavy metals, and receive a second survey result comprising a response to a second survey comprising a plurality of questions about each of the nutritional minerals; and
a recommendation test decision part configured to input the companion animal information and the first survey result as input values into a first machine-learning model that has been previously learned, obtain a first possibility that an abnormality related to the harmful heavy metals exits from the first machine-learning model, input the companion animal information and the second survey result as input values into a second machine-learning model that has been previously learned, obtain a second possibility that an abnormality related to the nutritional minerals exists from the second machine-learning model, and determine a test to be recommended to the user among a first test, second test and third test that have been preset based on the first possibility and the second possibility,
wherein the first test tests a health status of the companion animal related to the nutritional minerals,
the second test tests a health status of the companion animal related to the harmful heavy metals, and
the third test tests a health status of the companion animal related to the nutritional minerals and the harmful heavy metals.

4. The apparatus according to claim 3, wherein the solution analysis part:
inputs information on the age, neutering status, weight, personality, species, breed and body shape comprised in the companion animal information as input values into a third machine-learning model that has been previously learned and obtains the safe level of each of the harmful heavy metals from the third machine-learning model, and
inputs information on the age, neutering status, weight, personality, species, breed and body shape comprised in the companion animal information as input values into a fourth machine-learning model that has been previously learned and obtains the standard range of each of the nutritional minerals from the fourth machine-learning model.

5. The apparatus according to claim 1, wherein the nutritional minerals comprise calcium, sodium, potassium, phosphorus, magnesium, copper, zinc, iron, manganese, chromium and selenium, and the harmful heavy metals comprise mercury, arsenic, cadmium, lead, aluminum, nickel and uranium, and
the hair analysis result comprises:
a plurality of first indices comprising a ratio of the mineral level of the magnesium to the mineral level of the calcium, a ratio of the mineral level of the phosphorus to the mineral level of the calcium, a ratio of the mineral level of the potassium to the mineral level of the sodium, a ratio of the mineral level of the copper to the mineral level of the zinc, a ratio of the mineral level of the magnesium to the mineral level of the sodium and a ratio of the mineral level of the potassium to the mineral level of the calcium; and
a plurality of second indices comprising a ratio of the heavy metal level of the mercury to the mineral level of the zinc, a ratio of the heavy metal level of the mercury to the mineral level of the selenium, a ratio of the heavy metal level of the lead to the mineral level of the calcium, a ratio of the heavy metal level of the lead to the mineral level of the iron, a ratio of the heavy metal level of the lead to the mineral level of the zinc and a ratio of the heavy metal level of the cadmium to the mineral level of the zinc.
